(19) United States
(12) Patent Application Publication
(10) Pub. No.: US 2019/0201458 A1
(43) Pub. Date: Jul. 4, 2019

(54) MICROBIAL STRAINS AND THEIR USE IN ANIMALS

(71) Applicant: DUPONT NUTRITION BIOSCIENCES APS, Copenhagen (DK)

(72) Inventors: Stephane Frouel, Rennes (FR); Christopher Kromm, Richmond, MN (US)

(21) Appl. No.: 16/357,835

(22) Filed: Mar. 19, 2019

Related U.S. Application Data
(62) Division of application No. 14/512,925, filed on Oct. 13, 2014, now abandoned.
(60) Provisional application No. 61/745,324, filed on Dec. 21, 2012.
(60) Provisional application No. 61/623,512, filed on Apr. 12, 2012.

(51) Int. Cl. A61K 35/741; A61K 35/742; A23K 50/80; A23K 10/18; A61K 35/747; C12N 1/20

(52) U.S. Cl. CPC A61K 35/741; A61K 35/742; A23K 50/80; A23Y 2220/73; A61K 35/747; C12N 1/20; A23Y 2220/33; A23K 10/18

(57) ABSTRACT

Bacillus and Lactobacillus strains and methods that are useful for improving the performance of aquatic animals. The invention also discloses Bacillus and Lactobacillus strains and methods that are useful for inhibiting or slowing the growth of a pathogenic agent in an aquatic animal.

### CROSS-REFERENCE TO RELATED APPLICATIONS

This application claims the benefit of U.S. Provisional Application No. 61/623,512, filed Apr. 12, 2012 and U.S. Provisional Application No. 61/745,324, filed Dec. 21, 2012, the disclosures of which are hereby incorporated by reference.

### FIELD OF THE INVENTION

This invention is in the field of aquaculture. More specifically, this invention pertains to Bacillus and Lactobacillus strains that provide benefits to aquatic animals and methods of using these strains.

### DESCRIPTION OF THE RELATED ART

Aquaculture is an increasingly prevalent production system for providing fish and crustaceans for the human diet. Shrimp aquaculture has become a global industry with an annual retail value of billions of dollars. White shrimp (Penaeus vannamei) is one of major aquaculture species in the world. Shrimp farmers are highly interested in solutions that can improve water quality, production performance and survival rate.

In addition, diseases caused by pathogenic agents such as White Spot Syndrome virus (WSSv) and Vibrio species continually decimate shrimp farming industries in parts of Asia and South America. These losses lead to billions of dollars of economic loss and a decrease in productivity. Due to food safety and environmental concerns, the use of antibiotics is decreasing in shrimp aquaculture.

There is therefore a strong need in the aquaculture industry for antibiotic-free solutions to improve water quality, production performance, survival rate, and resistance to pathogenic agents in aquatic animals. In view of the foregoing, it would be desirable to provide Bacillus and Lactobacillus strains that provide benefits to aquatic animals and methods of using these strains.

### BRIEF DESCRIPTION OF THE DRAWINGS

Exemplary embodiments of the invention are illustrated in the accompanying drawings.

FIG. 1 is a schematic drawing showing the stages of development in shrimp from the larvae, post larvae, and juvenile stages.

FIG. 2A is a graph showing the survival, body weight gain (BWG), length, and feed intake responses in post larvae shrimp in response to administration of Bacillus and Lactobacillus compositions.

FIG. 2B are pictures of post larvae shrimp that show the size of the shrimp in response to administration of Bacillus and Lactobacillus compositions.

FIG. 3 is a graph showing the survival, body weight gain, feed conversion ratio (FCR), and feed intake responses in juvenile shrimp in response to administration of Bacillus and Lactobacillus compositions.

FIG. 4 are pictures showing the histology of the villi in shrimp that were administered Bacillus and Lactobacillus compositions, as compared to a control that was not treated with either composition.

FIG. 5 is a graph showing the Vibrio concentration in the shrimp gut in shrimp that were administered Bacillus and Lactobacillus compositions, as compared to a control that was not treated with either composition.

### SUMMARY OF THE INVENTION

The present invention provides isolated Bacillus and Lactobacillus strains, compositions comprising such Bacillus and Lactobacillus strains, methods of administering the strains to animals, animal feed or feed additive compositions comprising the strains, and methods of producing the strains.

In one embodiment, the invention provides one or more isolated Bacillus strains selected from the group consisting of B. subtilis, B. licheniformis, B. pumilus, B. coagulans, B. amyloliquefaciens, B. stearothermophilus, B. brevis, B. alkalophilus, B. clausii, B. halodurans, B. megaterium, B. circulans, B. lautus, B. thuringiensis and B. lentus. In another embodiment, the invention provides one or more isolated Lactobacillus strains selected from the group consisting of L. helveticus, L. amylovorus, L. curvatus, L. cellobiosus, L. amylolyticus, L. alimentarius, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae, L. sanfranciscensis, L. paracasei, L. casei, L. acidophilus, L. buchnerii, L. farciminis, L. rhamnosus, L. reuteri, L. fermentum, L. brevis, L. lactis, L. plantarum, L. sakei or L. salviarium strains.

In particular embodiments, the invention provides one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

In another embodiment, the invention provides a composition comprising one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

In some embodiments, the invention provides a composition comprising a Bacillus pumilis 3064 strain, a Bacillus subtilis BS 2084 (NRRL B-50013) strain, and a Bacillus subtilis BS15 Ap4 (ATCC PTA-6507) strain. In another embodiment, the invention provides a composition comprising a Bacillus pumilis 119 (NRRL B-50796) strain, a Bacillus subtilis BS 2084 (NRRL B-50013) strain, and a Bacillus subtilis BS15 Ap4 (ATCC PTA-6507) strain. In another embodiment, the invention provides a composition comprising a Bacillus subtilis 1013 (NRRL B-50509) strain, a Bacillus subtilis BS918 (NRRL B-50508) strain, and a Bacillus subtilis BS3BP5 (ATCC PTA-50510) strain. In another embodiment, the invention provides a composition comprising a Bacillus licheniformis 842 (NRRL B-50516) strain, a Bacillus subtilis BS27 (NRRL B-50105) strain, and a Bacillus licheniformis BL21 (ATCC PTA-50134) strain. In other embodiments, the invention provides a composition comprising a Bacillus subtilis 3A-P4 (ATCC PTA-6506), strain, a Bacillus subtilis BS15 Ap4 (ATCC PTA-6507) strain, and a Bacillus subtilis 22C-P1 (ATCC PTA-6508) strain.

In another embodiment, the invention provides a method comprising administering to an animal an effective amount of a composition comprising one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

In some embodiments, the methods described herein comprise administering to an animal an effective amount of a composition comprising a Bacillus pumilis 3064 strain. a Bacillus subtilis BS 2084 (NRRL B-50013) strain, and a Bacillus subtilis BS15 Ap4 (ATCC PTA-6507) strain. In some embodiments, the methods described herein comprise administering to an animal an effective amount of a composition comprising a Bacillus pumilis 119 (NRRL B-50796) strain, a Bacillus subtilis BS 2084 (NRRL B-50013) strain, and a Bacillus subtilis BS15 Ap4 (ATCC PTA-6507) strain. In some embodiments, the methods described herein comprise administering to an animal an effective amount of a composition comprising a Bacillus subtilis 1013 (NRRL B-50509) strain, a Bacillus subtilis BS918 (NRRL B-50508) strain, and a Bacillus subtilis BS3BP5 (ATCC PTA-50510) strain. In some embodiments, the methods described herein comprise administering to an animal an effective amount of a composition comprising a Bacillus licheniformis 842 (NRRL B-50516) strain, a Bacillus subtilis BS27 (NRRL B-50105) strain, and a Bacillus licheniformis BL21 (ATCC PTA-50134) strain. In some embodiments, the methods described herein comprise administering to an animal an effective amount of a composition comprising a Bacillus subtilis 3A-P4 (ATCC PTA-6506), strain, a Bacillus subtilis BS15 Ap4 (ATCC PTA-6507) strain, and a Bacillus subtilis 22C-P1 (ATCC PTA-6508) strain.

In any of the embodiments described herein, upon administration to the animal, the strain provides at least one of the following benefits in or to the animal when compared to an animal not administered the strain: (a) increased survival, (b) increased body weight gain (either or both of average daily weight gain or total weight gain), (c) increased feed intake, (d) increased length, (e) increased feed conversion, (f) increased villi length and/or density, (g) increased resistance to low salinity, (h) increased resistance to high salinity, (i) increased resistance to high temperature, (j) increased resistance to low temperature, (k) increased resistance to formalin, (l) increased survival in response a pathogenic agent, or (m) mortality. The present invention provides benefits against stress and pathogenic infection in an animal. In some embodiments, the present invention provides increased survival against a pathogenic agent, such as for example, White Spot Syndrome virus or Vibrio spp. (e.g., Vibrio harveyi). In other embodiments, the invention provides increased resistance to high or low temperatures, or high or low salinity. In certain embodiments, the animal is exposed to high or low temperatures, high or low salinity, white spotted syndrome virus, or Vibrio spp.

In any embodiments described herein, the animal is a shrimp. In some embodiments, the shrimp is a larvae, post-larvae, or juvenile shrimp. Shrimp that are used in the embodiments described herein include all variety and species of shrimp, including by way of example and not limitation, Litopenaeus, Farfantepenaeus, and Penaeus. Penaeus spp. include, without limitation, Penaeus stylirostris, Penaeus vannamei, Penaeus monodon, Penaeus chinensis, Penaeus occidentalis, Penaeus californiensis, Penaeus semisulcatus, Penaeus monodon, Penaeus esculentu, Penaeus setiferus, Penaeus japonicus, Penaeus aztecus, Penaeus duorarum, Penaeus indicus, and Penaeus merguiensis. In particular environments, the shrimp is Penaeus vannamei.

In certain embodiments, when a strain described herein is administered to an animal, the strain provides an improvement in at least one of the benefits described herein by at least 2% compared to an untreated control. The provided strains can be administered at any concentration effective to improve at least one of the benefits described herein. In some embodiments, the strain(s) is/are administered at about 1×10⁵ to about 1×10¹¹ CFU/animal/day.

In another embodiment, the invention provides an animal feed or feed additive composition, comprising one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

In some embodiments, the invention provides an animal feed or feed additive composition comprising a Bacillus pumilis 3064 strain, a Bacillus subtilis BS 2084 (NRRL B-50013) strain, and a Bacillus subtilis BS15 Ap4 (ATCC PTA-6507) strain. In some embodiments, the invention provides an animal feed or feed additive composition comprising a Bacillus pumilis 119 (NRRL B-50796) strain, a Bacillus subtilis BS 2084 (NRRL B-50013) strain, and a Bacillus subtilis BS15 Ap4 (ATCC PTA-6507) strain. In some embodiments, the invention provides an animal feed or feed additive composition comprising a Bacillus subtilis 1013 (NRRL B-50509) strain, a Bacillus subtilis BS918 (NRRL B-50508) strain, and a Bacillus subtilis BS3BP5 (ATCC PTA-50510) strain. In some embodiments, the invention provides an animal feed or feed additive composition comprising a Bacillus licheniformis 842 (NRRL B-50516) strain, a Bacillus subtilis BS27 (NRRL B-50105) strain, and a Bacillus licheniformis BL21 (ATCC PTA-50134) strain. In some embodiments, the invention provides an animal feed or feed additive composition comprising a Bacillus subtilis 3A-P4 (ATCC PTA-6506) strain, a Bacillus subtilis BS15 Ap4 (ATCC PTA-6507) strain, and a Bacillus subtilis 22C-P1 (ATCC PTA-6508) strain. In some embodiments, the one or more strains described herein are supplemented in an animal feed or feed additive composition in an amount of 10 to 2000 grams per ton of feed. In some embodiments, the one or more strains described herein are supplemented in an animal feed or feed additive composition in an amount of 50, 100, 250, 500, or 1000 grams per ton of feed.

In some embodiments, the invention provides a method of producing one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof, comprising: (a) growing, in a liquid broth, a culture including the one or more strain(s); and (b) separating the one or more strains from the liquid broth. In some embodiments, the method further comprises freeze drying the isolated strain and adding the freeze-dried strain to a carrier. In other embodiments, the method further comprises retaining the liquid broth after the strain has been separated from it to generate a supernatant.

### DETAILED DESCRIPTION

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

In accordance with the present invention, there may be employed conventional molecular biology and microbiology within the skill of the art. Such techniques are explained fully in the literature. See, e.g., Sambrook, Fritsch & Maniatis, Molecular Cloning: A Laboratory Manual, Third Edition (2001) Cold Spring Harbor Laboratory Press, Cold Spring Harbor, N.Y.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a recited range is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

The inventors have found that certain microbial strains are useful for improving the performance in aquatic animals. In addition, the inventors have found that certain microbial strains are useful for inhibiting or slowing the growth of pathogens in aquatic animals or increasing the resistance in aquatic animals to stress.

Described herein are Bacillus and Lactobacillus strains that have positive effects on the health of aquatic animals. Preferred Bacillus and Lactobacillus strains will now be described that are useful in aquatic animals. This example is not intended to limit the invention to Bacillus and Lactobacillus strains usable only in aquatic animals.

In one embodiment, the Bacillus and Lactobacillus strains are useful for improving the performance of an aquatic animal. As used herein, “performance” refers to one or more of the following parameters in an aquatic animal, such as a shrimp: (a) increased survival, (b) increased body weight gain (either or both of average daily weight gain or total weight gain), (c) increased feed intake, (d) increased length, (e) feed conversion, which includes both feed:gain and gain:feed, (f) increased villi length and/or density, (g) increased resistance to low salinity, (h) increased resistance to high salinity, (i) increased resistance to high temperature, (j) increased resistance to low temperature, (k) increased resistance to formalin, (1) increased survival in response to a pathogenic agent, such as WSSv or Vibrio spp., (m) mortality, and other measurements known in the art.

“An improvement in performance” or “improved performance” as used herein, means an improvement in at least one of the parameters listed under the performance definition. The improved performance is measured relative to a control animal. Control animals described herein are animals (e.g., shrimp) which have not been administered the Bacillus and/or Lactobacillus composition.

The present application provides methods of administering an effective amount of one or more Bacillus or Lactobacillus strains to an aquatic animal, such as a shrimp. In one embodiment, the methods improve performance of an aquatic animal. Thus, it may be economical for an aquaculture producer to routinely administer one or more Bacillus or Lactobacillus strains, either individually or in combination with other Bacillus or Lactobacillus strains, not only to treat and prevent disease, but also to improve performance.

In another embodiment, administration of one or more Bacillus or Lactobacillus stains inhibit or slow the growth of pathogenic microbes. For instance, administration of one or more Bacillus or Lactobacillus stains inhibit or slow the growth of White Spot Syndrome virus (WSSv) or Vibro spp. The methods may also be used to reduce or prevent disease associated with WSSv or Vibro sp. in aquatic animals that are not currently infected with such pathogens. By inhibiting or slowing the growth of a pathogenic agent, an aquatic animal described herein will demonstrate an improvement in survival when exposed to the pathogenic agent, or otherwise demonstrate an improvement in performance as described herein.

In another embodiment, administration of one or more Bacillus or Lactobacillus strains allows the aquatic animal to have an increased resistance to stress. For instance, administration of one or more Bacillus or Lactobacillus stains allows the aquatic animal to have an increased resistance to high or low salinity, high or low temperatures, or high or low formalin exposure. By increasing the animal's resistance to stress, an aquatic animal described herein will demonstrate an improvement in survival when exposed to the stress, or otherwise demonstrate an improvement in performance as described herein.

Methods of administering one or more Bacillus or Lactobacillus strains to an aquatic animal are also provided. Such methods may include feeding the one or more Bacillus or Lactobacillus strains to an aquatic animal such as a shrimp. The strain(s) may be fed during the larval stage, post-larval stage, juvenile stage, or any other stage of growth of the animal. Bacillus strains, in particular, have many qualities that make them useful for compositions that are ingested by animals. For example, Bacillus strains produce extracellular enzymes, such as proteases, amylases, and cellulases. In addition, Bacillus strains produce antimicrobial factors, such as gramicidin, subtilin, bacitracin, and polymyxin. Furthermore, Bacillus strains are spore-formers and thus are stable. Additionally, several species of Bacillus have GRAS status, i.e., they are generally recognized as safe. Bacillus species are the only spore-formers that are considered GRAS.

The Bacillus and Lactobacillus strains described herein inhibit or slow the growth of one or more pathogens in an aquatic animal. For instance, pathogens within the scope of the invention include a wide variety of agents that specifically infect mariculture. Pathogens include viral or bacterial pathogens as well as toxins produced by algae such as, for example, dinoflagellates. These pathogens include, by way of example and not limitations, White Spot Syndrome Virus (WSSv), Taura Syndrome Virus (TSV), Yellow Head Virus (YHV), species of Vibrio (including V. anguillarum and V. ordalii, Vibrio salmonicida, Vibrio harveyi), causative agents and virus for infectious hypodermal and haematopoietic necrosis (IHHN) and IHHNV, causative agent for run-deformity syndrome or RDS of Penaeus vannamei, Baculo-like viruses, Infectious Pancreatic Necrosis Virus (IPNV), Hirame rhabdovirus (HIRRV), the Yellowtail Ascites Virus (YAV), Striped Jack Nervous Necrosis Virus (SJNNV), Irido, Aeromonos hydrophila, Aeromonos salmonicida, Serratia liquefaciens, Yersnia ruckeri type I, Infectious salmon anaemia (USA) virus, Pancreas Disease (PD), Viral Hemorrhagic Septicemia (VHS), Rennibacterium salmoninarum, Aeromonas salmonicida, Aeromonas hydrophila, species of Pasteurella (including P. piscicida), species of Yersinia, species of Streptococcus, Edwardsiella tarda and Edwardsiella ictaluria; the viruses causing viral hemorrhagic septicemia, infectious pancreatic necrosis, viremia of carp, channel catfish virus, grass carp hemorrhagic virus, nodaviridae such as nervous necrosis virus, infectious salmon anaemia virus; and the parasites Ceratomyxa shasta, Ichthyophthirius multifillius, Cryptobia salmositica, Lepeophtherius salmonis, Tetrahymena species, Trichodina species and Epistylus species, dinoflagellates toxins including toxins causing Diaarhetic Shellfish Poisoning (DSP), Paralytic Shellfish Poisoning (PSP), Neurotoxin poisoning (NSP) and Ciguatera, and many more, all of which cause serious damage in aquaculture. In one embodiment, the Bacillus and Lactobacillus strains or the invention inhibit or slow the growth of WSSv or a Vibrio spp. in an aquatic animal. In another embodiment, the Bacillus and Lactobacillus strains or the invention inhibit or slow the growth of WSSv or Vibrio harveyi in an aquatic animal. Multiple Bacillus strains can be combined for control of various pathogens such as those above. Bacillus strains found useful for uses described herein include, but are not limited to, B. subtilis, B. licheniformis, B. pumilus, B. coagulans, B. amyloliquefaciens, B. stearothermophilus, B. brevis, B. alkalophilus, B. clausii, B. halodurans, B. megaterium, B. circulans, B. lautus, B. thuringiensis and B. lentus strains. In at least some embodiments, the Bacillus strain(s) is (are) Bacillus pumilis 3064, Bacillus subtilis BS 2084, Bacillus subtilis BS15 Ap4, Bacillus subtilis AGTP BS3BP5, Bacillus subtilis AGTP BS442, Bacillus subtilis AGTP BS521, Bacillus subtilis AGTP BS918, Bacillus subtilis AGTP BS1013, Bacillus pumilis 119, Bacillus subtilis 3A-P4, Bacillus subtilis 22C-P1, Bacillus licheniformis 842, Bacillus subtilis BS27, Bacillus licheniformis BL21, and Bacillus subtilis AGTP BS1069. In at least some embodiments, the B. pumilus strain is Bacillus pumilus AGTP BS 1068. In one embodiment, the Bacillus strains used in the invention is a combination of Bacillus pumilis 3064, Bacillus subtilis BS 2084, and Bacillus subtilis BS15 Ap4. In another embodiment, the Bacillus strains used in the invention is a combination of Bacillus pumilis 119, Bacillus subtilis BS 2084, and Bacillus subtilis BS15 Ap4. In another embodiment, the Bacillus strains used in the invention is a combination of Bacillus subtilis BS1013, Bacillus subtilis BS918, and Bacillus subtilis BS3BP5. In another embodiment, the Bacillus strains used in the invention is a combination of Bacillus subtilis 3A-P4, Bacillus subtilis 15A-P4, and Bacillus subtilis 22C-P1. In another embodiment, the Bacillus strains used in the invention is a combination of Bacillus licheniformis 842, Bacillus subtilis BS27, and Bacillus licheniformis BL21.

These strains were deposited by Danisco USA, Inc. of Waukesha, Wis. at the Agricultural Research Service Culture Collection (NRRL), 1815 North University Street, Peoria, Ill., 61604. The dates of original deposits and accession numbers are as follows: Bacillus subtilis AGTP BS3BP5, May 13, 2011 (NRRL B-50510), Bacillus subtilis AGTP BS442, Aug. 4, 2011 (NRRL B-50542), Bacillus subtilis AGTP BS521, Aug. 4, 2011 (NRRL B-50545), Bacillus subtilis AGTP BS918, May 13, 2011 (NRRL B-50508), Bacillus subtilis AGTP BS1013, May 13, 2011 (NRRL B-50509), Bacillus pumilus AGTP BS 1068, Aug. 4, 2011 (NRRL B-50543), and Bacillus subtilis AGTP BS1069, Aug. 4, 2011 (NRRL B-50544). Bacillus subtilis BS 2084 (NRRL B-50013) was deposited on Mar. 8, 2007 at the Agricultural Research Service Culture Collection (NRRL), 1815 North University Street, Peoria, Ill., 61604. All of the deposits were made under the provisions of the Budapest Treaty on the International Recognition of the Deposit of Microorganisms for the Purposes of Patent Procedure.

The following strains were deposited by Agtech Products, Inc. of Waukesha, Wis. at American Type Culture Collection (ATCC) 10801 University Blvd., Manassas, Va. 20110. The dates of original deposits and accession numbers are as follows: Bacillus subtilis 3A-P4, Jan. 12, 2005 (ATCC PTA 6506), Bacillus subtilis BS15-AP4, Jan. 12, 2005 (ATCC PTA-6507), Bacillus subtilis 22C-P1, Jan. 12, 2005 (ATCC PTA-6508).

Bacillus licheniformis 842 was deposited by Danisco USA of Waukesha, Wis. at Agricultural Research Service Culture Collection (NRRL) on May 20, 2011 (NRRL B-50516). Bacillus subtilis BS27 was deposited by AgTech Inc. of Waukesha, Wis. at Agricultural Research Service Culture Collection (NRRL) on Jan. 24, 2008 (NRRL B-50105). Bacillus licheniformis BL21 was deposited by AgTech Products, Inc. of Waukesha, Wis. at Agricultural Research Service Culture Collection (NRRL) on Apr. 15, 2008 (NRRL B-50134). Bacillus pumilus BP119 was deposited by DuPont Nutrition Biosciences ApS of Copenhagen, Denmark at Agricultural Research Service Culture Collection (NRRL) on Dec. 18, 2012 (NRRL B-50796) and is also commercially available from Genesis Biosciences (Lawrenceville, Ga.).

Any Bacillus derivative or variant is also included and is useful in the methods described and claimed herein. In some embodiments, strains having all the characteristics of Bacillus pumilis 3064, Bacillus subtilis BS 2084, Bacillus subtilis BS15 Ap4, Bacillus subtilis AGTP BS3BP5, Bacillus subtilis AGTP BS442, Bacillus subtilis AGTP BS521, Bacillus subtilis AGTP BS918, Bacillus subtilis AGTP BS1013, Bacillus pumilis 119, Bacillus subtilis 3A-P4, Bacillus subtilis 22C-P1, Bacillus licheniformis 842, Bacillus subtilis BS27, Bacillus licheniformis BL21, Bacillus pumilus AGTP BS 1068, and Bacillus subtilis AGTP BS1069 are also included and are useful in the methods described and claimed herein.

In certain embodiments, any derivative or variant of Bacillus pumilis 3064, Bacillus subtilis BS 2084, Bacillus subtilis BS15 Ap4, Bacillus subtilis AGTP BS3BP5, Bacillus subtilis AGTP BS442, Bacillus subtilis AGTP BS521, Bacillus subtilis AGTP BS918, Bacillus subtilis AGTP BS1013, Bacillus pumilis 119, Bacillus subtilis 3A-P4, Bacillus subtilis 22C-P1, Bacillus licheniformis 842, Bacillus subtilis BS27, Bacillus licheniformis BL21, Bacillus pumilus AGTP BS 1068, and Bacillus subtilis AGTP BS1069 are also included and are useful in the methods described and claimed herein.

The genetic profiles of strains Bacillus pumilus 3064 and Bacillus pumilis 119 (NRRL B-50796) were compared using standard RAPD pattern analysis and Sanger sequencing of Chaperonin-60 universal target (cpn60) and 16S rDNA. Based on RAPD banding patterns and cpn60 and 16S rDNA sequence analysis of five replicate samples, Bacillus pumilus 3064 and Bacillus pumilis 119 (NRRL B-50796) were determined to be genetically equivalent. Accordingly, as used herein, Bacillus pumilus 3064 and Bacillus pumilis 119 are used interchangeably.

Lactobacillus strains found useful for uses described herein include, but are not limited to, L. helveticus, L. amylovorus, L. curvatus, L. cellobiosus, L. amylolyticus, L. alimentarius, L. aviaries, L. crispatus, L. curvatus, L. gallinarum, L. hilgardii, L. johnsonii, L. kefiranofaecium, L. kefiri, L. mucosae, L. panis, L. pentosus, L. pontis, L. zeae, L. sanfranciscensis, L. paracasei, L. casei, L. acidophilus, L. buchnerii, L. farciminis, L. rhamnosus, L. reuteri, L. fermentum, L. brevis, L. lactis, L. plantarum, L. sakei or L. salviarium strains. In at least some embodiments, the Lactobacillus strains are Lactobacillus farcimins CNCM-I-3699, Lactobacillus rhamnosus CNCM-I-3698, or combinations thereof. Both Lactobacillus farcimins CNCM-I-3699 and Lactobacillus rhamnosus CNCM-I-3698 were deposited in the National Micro-organism Collection of Pasteur Institute (CNCM, Paris).

Any Lactobacillus derivative or variant is also included and is useful in the methods described and claimed herein. In some embodiments, strains having all the characteristics of Lactobacillus farcimins CNCM-I-3699 or Lactobacillus rhamnosus CNCM-I-3698 are also included and are useful in the methods described and claimed herein.

In certain embodiments, any derivative or variant of Lactobacillus farcimins CNCM-I-3699 or Lactobacillus rhamnosus CNCM-I-3698 are also included and are useful in the methods described and claimed herein.

As used herein, a “variant” has at least 80% identity of genetic sequences with the disclosed strains using random amplified polymorphic DNA polymerase chain reaction (RAPD-PCR) analysis. The degree of identity of genetic sequences can vary. In some embodiments, the variant has at least 85%, 90%, 95%, 96%, 97%, 98%, or 99% identity of genetic sequences with the disclosed strains using RAPD-PCR analysis. Six primers that can be used for RAPD-PCR analysis include the following: Primer 1 (5′-GGTGCGGGAA-3′) (SEQ ID NO:1), PRIMER 2 (5′-GTTTCGCTCC-3′) (SEQ ID NO:2), PRIMER 3 (5′-GTAGACCCGT-3′) (SEQ ID NO:3), PRIMER 4 (5′-AAGAGCCCGT-3′) (SEQ ID NO:4, PRIMER 5 (5′-AACGCGCAAC-3′) (SEQ ID NO: 5), PRIMER 6 (5′-CCCGTCAGCA-3′) (SEQ ID NO: 6). RAPD analysis can be performed using Ready-to-Go′ RAPD Analysis Beads (Amersham Biosciences, Sweden), which are designed as pre-mixed, pre-dispensed reactions for performing RAPD analysis.

### Preparation and Feeding of Direct-Fed Microbials

To prepare DFMs described herein, the strains can be grown in a liquid nutrient broth. For Bacillus strains, the growth is preferably to a level at which the highest number of spores are formed. In one embodiment, the strains are grown to an optical density (OD) where the yield is at least 10⁷-10⁹ colony forming units (CFU) per ml of culture. The strains of the present invention are produced by fermentation of the bacterial strains. Fermentation is started by scaling-up a seed culture. This involves repeatedly and aseptically transferring the culture to a larger and larger volume to serve as the inoculum for the fermentation, which is carried out in large stainless steel fermentors in medium containing proteins, carbohydrates, and minerals necessary for optimal growth. A non-limiting exemplary medium is Trypticase Soy Broth. After the inoculum is added to the fermentation vessel, the temperature and agitation are controlled to allow maximum growth. Once the culture reaches a maximum population density, the culture is harvested by separating the cells from the fermentation medium. This is commonly done by centrifugation. The supernatant can be used in the methods described herein. The count of the culture can then be determined.

In at least some embodiments, the bacteria are pelleted. In at least some embodiments, the bacteria are freeze-dried. In at least some embodiments, the bacteria are mixed with a carrier. However, it is not necessary to freeze-dry the strains before using them. The strains can also be used with or without preservatives, and in concentrated, unconcentrated, or diluted form.

The count of the culture can then be determined. CFU or colony forming unit is the viable cell count of a sample resulting from standard microbiological plating methods. The term is derived from the fact that a single cell when plated on appropriate medium will grow and become a viable colony in the agar medium. Since multiple cells may give rise to one visible colony, the term colony forming unit is a more useful unit measurement than cell number.

The count of the bacteria is important when combined with a carrier. In one embodiment, at the time of manufacture of the composition, the count is at least about 1.0×10⁶-1.0×10¹² CFU/g. The counts may be increased or decreased, however, from these base numbers and still have complete efficacy. For example, the count at the time of manufacture of the composition can be at least about 1.0×10³, 1.0×10⁴, 1.0×10⁵, 1.0×10⁶, 1.0×10⁷, 1.0×10⁸, 1.0×10⁹, 1.0×10¹⁰, 1.0×10¹¹, 1.0×10¹², 1.0×10¹³, 1.0×10¹⁴, or 1.0×10¹⁵ CFU/g.

A composition including one or more strain(s) described herein is provided. The composition can be fed to an aquatic animal as a direct-fed microbial (DFM). One or more carrier(s) or other ingredients can be added to the DFM. The DFM may be presented in various physical forms, for example, as a top dress, as a water soluble concentrate for use as a liquid drench or to be added to a milk replacer, gelatin capsule, or gels. In one embodiment of the top dress form, freeze-dried lactic acid bacteria fermentation product is added to a carrier, such as whey, maltodextrin, sucrose, dextrose, limestone (calcium carbonate), rice hulls, yeast culture, dried starch, and/or sodium silico aluminate. In one embodiment of the water soluble concentrate for a liquid drench or milk replacer supplement, freeze-dried lactic acid bacteria fermentation product is added to a water soluble carrier, such as whey, maltodextrin, sucrose, dextrose, dried starch, sodium silico aluminate, and a liquid is added to form the drench or the supplement is added to milk or a milk replacer. In one embodiment of the gelatin capsule form, freeze-dried lactic acid bacteria fermentation product is added to a carrier, such as whey, maltodextrin, sugar, limestone (calcium carbonate), rice hulls, yeast culture dried starch, and/or sodium silico aluminate. In one embodiment, the lactic acid bacteria and carrier are enclosed in a degradable gelatin capsule. In one embodiment of the gels form, freeze-dried lactic acid fermentation product is added to a carrier, such as vegetable oil, sucrose, silicon dioxide, polysorbate 80, propylene glycol, butylated hydroxyanisole, citric acid, ethoxyquin, and/or artificial coloring to form the gel.

The strain(s) may optionally be admixed with a dry formulation of additives including but not limited to growth substrates, enzymes, sugars, carbohydrates, extracts and growth promoting micro-ingredients. The sugars could include the following: lactose; maltose; dextrose; malto-dextrin; glucose; fructose; mannose; tagatose; sorbose; raffinose; and galactose. The sugars range from 50-95%, either individually or in combination. The extracts could include yeast or dried yeast fermentation solubles ranging from 5-50%. The growth substrates could include: trypticase, ranging from 5-25%; sodium lactate, ranging from 5-30%; and, Tween 80, ranging from 1-5%. The carbohydrates could include mannitol, sorbitol, adonitol and arabitol. The carbohydrates range from 5-50% individually or in combination. The micro-ingredients could include the following: calcium carbonate, ranging from 0.5-5.0%; calcium chloride, ranging from 0.5-5.0%; dipotassium phosphate, ranging from 0.5-5.0%; calcium phosphate, ranging from 0.5-5.0%; manganese proteinate, ranging from 0.25-1.00%; and, manganese, ranging from 0.25-1.0%.

The culture(s) and carrier(s) (where used) can be added to a ribbon or paddle mixer and mixed for about 15 minutes, although the timing can be increased or decreased. The components are blended such that a uniform mixture of the cultures and carriers result. The final product is preferably a dry, flowable powder. The strain(s) can then be added to animal feed or a feed premix, added to an animal's water, or administered in other ways known in the art. A feed for an animal can be supplemented with one or more strain(s) described herein or with a composition described herein.

The strains can be administered in an effective amount to animals, which include, but are not limited to aquatic animals. Aquatic animals include vertebrates, invertebrates, arthropods, fish, mollusks, including, by way of example and not limitation, shrimp (e.g., penaeid shrimp, brine shrimp, freshwater shrimp, etc), crabs, oysters, scallop, prawn clams, cartilaginous fish (e.g., bass, striped bass, tilapia, catfish, sea bream, rainbow trout, zebrafish, red drum, salmonids, carp, catfish, yellowtail, carp, etc), crustaceans, among others. Shrimp includes all variety and species of shrimp, including by way of example and not limitation, Litopenaeus, Farfantepenaeus, and Penaeus. Penaeus spp. include, without limitation, Penaeus stylirostris, Penaeus vannamei, Penaeus monodon, Penaeus chinensis, Penaeus occidentalis, Penaeus californiensis, Penaeus semisulcatus, Penaeus monodon, Penaeus esculentu, Penaeus setiferus, Penaeus japonicus, Penaeus aztecus, Penaeus duorarum, Penaeus indicus, and Penaeus merguiensis, among others species of shrimp.

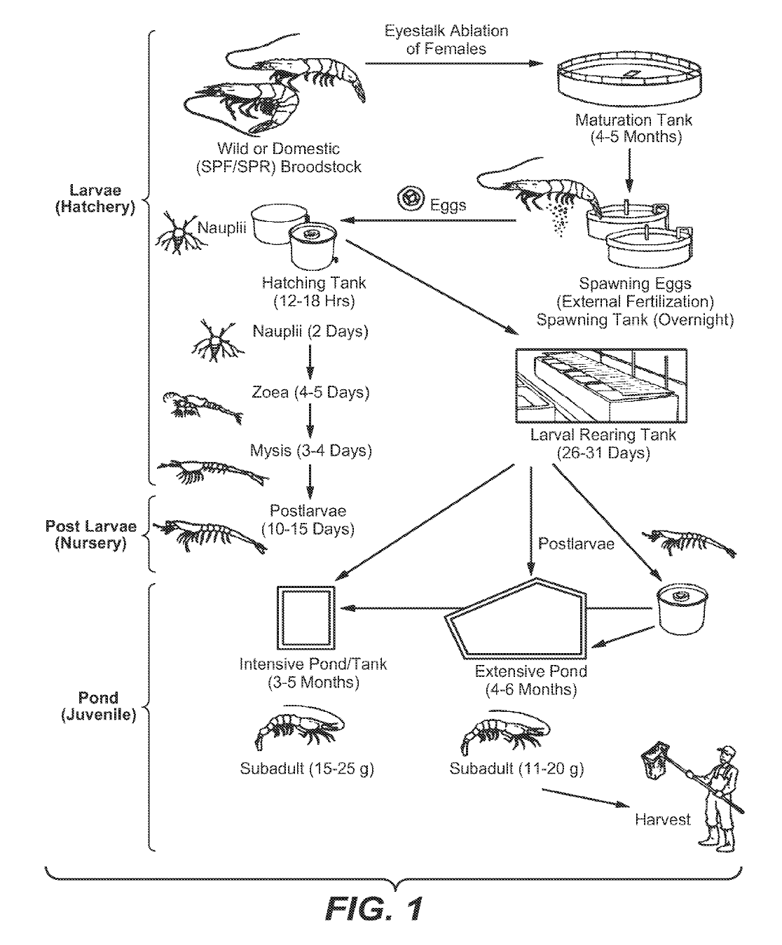

The Bacillus and Lactobacillus compositions described herein can be administered to an aquatic animal at any stage of growth. In some embodiments, the compositions are administered to shrimp during the larvae, post-larvae, or juvenile stage of growth. See e.g., FIG. 1.

By “administer,” is meant the action of introducing at least one strain and/or supernatant from a culture of at least one strain described herein to an aquatic animal. In some embodiments administration of the at least one strain is to the gastrointestinal tract of the animal. The administration can be by oral route. This administration can in particular be carried out by supplementing the feed intended for the animal with the at least one strain, the supplemented feed then being ingested by the animal. The administration can also be carried out using a stomach tube or any other way to make it possible to directly introduce the at least one strain into the animal's gastrointestinal tract. In some embodiments, administration of one or more strains to animals is accomplished by any convenient method, including adding the Bacillus or Lactobacillus strains to water that contacts the animal or that the animal ingests, by top dress, as a water soluble concentrate for use as a liquid drench, gelatin capsule, or gels. Bacillus strains preferably are administered as spores.

By “effective amount,” is meant a quantity of DFM and/or supernatant sufficient to allow improvement in performance of the animal, or to inhibit or slow growth of a pathogenic agent described herein. The amount of improvement can be measured as described herein or by other methods known in the art. These effective amounts can be administered to the animal by providing ad libitum access to feed containing the DFM. The DFM can also be administered in one or more doses.

In at least some embodiments, the improvement is by at least 2% compared to an untreated control. In certain embodiments, the improvement is by at least 5%, 10%, 15%, 20%, 25%, 30%, 35%, 40%, 45%, 50%, 55%, 60%, 65%, 70%, 75%, 80%, 85%, 90%, or 95%.

This effective amount can be administered to the animal in one or more doses. By “at least one strain,” is meant a single strain but also mixtures of strains comprising at least two strains of bacteria. In at least some embodiments, more than one of the strain(s) described herein is (are) combined. By “a mixture of at least two strains,” is meant a mixture of two, three, four, five, six or even more strains. In some embodiments of a mixture of strains, the proportions can vary from 1% to 99%. In certain embodiments, the proportion of a strain used in the mixture is at least 5%, 10%, 15%, 20%, 25%, 30%, 35%, 40%, 45%, 50%, 55%, 60%, 65%, 70%, 75%, 80%, 85%, 90%, or 95%. Other embodiments of a mixture of strains are from 25% to 75%. Additional embodiments of a mixture of strains are approximately 50% for each strain. When a mixture comprises more than two strains, the strains can be present in substantially equal proportions in the mixture or in different proportions.

For example, the strains can be combined in different ratios to determine the best ratio to improve animal performance or inhibit or slow the growth of a pathogenic agent. When used in combination, the following exemplary, non-limiting ratios of strains can be used: ⅓ each of three different strains; ¼ each of four different strains; ⅕ each of five different stains; 40% of a first strain, 40% of a second strain, and 20% of a third strain; 50% of a first strain, 25% of a second strain, and 25% of a third strain; 70% of a first strain, 20% of a second strain, and 10% of a third strain. Other combinations of strains can also be used. In addition, a combination having 50% more CFU per gram can be used to boost the amount of microorganism fed to the animal.

In some embodiments, when the bacteria are added to animal feed or to an animal feed additive, the amount that is added is at least about 10-20,000 grams per ton of feed. This amount can be increased or decreased, however, from this number and still have complete efficacy. For example, the amount of bacteria that are added can be 10, 25, 50, 100, 200, 300, 400, 500, 750, 1000, 1500, 2000, 2500, 3000, 4000, 5000, 10,000, 15,000, or 20,000 grams per ton of feed. Alternatively, the amount can be any amount in the range of 50-20,000 grams per ton of feed.

In some embodiments, the one or more Bacillus or Lactobacillus strain(s) is (are) added to an animal's feed at a rate of at least 1.0×10¹ CFU/animal/day. In another embodiment, the one or more Bacillus or Lactobacillus strain(s) is (are) added to an animal's feed at a rate of at least 1.0×10², 1.0×10³, 1.0×10⁴, 1.0×10⁵, 1.0×10⁶, 1.0×10⁷, 1.0×10⁸, 1.0×10⁹, 1.0×10¹⁰, 1.0×10¹¹, 1.0×10¹², 1.0×10¹³, 1.0×10¹⁴, or 1.0×10¹⁵ CFU/animal/day.

In some embodiments, the one or more Bacillus or Lactobacillus strain(s) is (are) added to an animal's feed at a rate of at least 1.0×10³ CFU per gram of feed. In another embodiment, the one or more Bacillus or Lactobacillus strain(s) is (are) added to an animal's feed at a rate of at least 1.0×10⁴, 1.0×10⁵, 1.0×10⁶, 1.0×10⁷, 1.0×10⁸, 1.0×10⁹, 1.0×10¹⁰, 1.0×10¹¹, 1.0×10¹² 1.0×10¹³, 1.0×10¹⁴, or 1.0×10¹⁵ CFU per gram of feed. In a particular embodiment, the Bacillus or Lactobacillus strain is added to an animal's feed at a rate of at least 2×10⁸-2×10⁹ CFU/g.

The DFM provided herein can be administered, for example, as the strain-containing culture solution, the strain-producing supernatant, or the bacterial product of a culture solution.

The DFM may be administered to the animal in one of many ways. For example, the strain(s) can be administered in a solid form, may be distributed in an excipient, preferably water, and directly fed to the animal, may be physically mixed with feed material in a dry form, or the strain(s) may be formed into a solution and thereafter sprayed onto feed material. The method of administration of the strain(s) to the animal is considered to be within the skill of the artisan.

When used in combination with a feed material the feed material can include corn, soybean meal, byproducts like distillers dried grains with solubles, rice hulls, calcium carbonate, mineral oil, seaweed meal, crushed soy, bran, silicon dioxide, calcium propionate, or vitamin/mineral supplement.

The time of administration is not crucial so long as an improvement is shown in one or more of the performance characteristics described herein, such as: (a) increased survival, (b) increased body weight gain (either or both of average daily weight gain or total weight gain), (c) increased feed intake, (d) increased length, (e) feed conversion, which includes both feed:gain and gain:feed, (f) increased villi length and/or density, (g) increased resistance to low salinity, (h) increased resistance to high salinity, (i) increased resistance to high temperature, (j) increased resistance to low temperature, (k) increased resistance to formalin, (1) increased survival in response to a pathogenic agent, such as WSSv or Vibrio spp., or (m) mortality. Administration is possible at any time with or without feed. However, the Bacillus or Lactobacillus composition is preferably administered with or immediately before feed.

Thus, in at least some embodiments, the effective amount of at least one strain of bacterium is administered to an animal by supplementing a feed intended for the animal with the effective amount of at least one strain of bacterium. As used herein, “supplementing,” means the action of incorporating the effective amount of bacteria provided herein directly into the feed intended for the animal. Thus, the animal, when feeding, ingests the bacteria provided herein.

A feed for an animal comprises at least one strain of bacterium described herein.

In at least some embodiments, a method comprising the step of administering to an aquatic animal an effective amount of the Bacillus or Lactobacillus compositions, one or more combination(s) of the Bacillus or Lactobacillus compositions, one or more supernatant(s) from a culture of the Bacillus or Lactobacillus compositions, feed including one or more Bacillus or Lactobacillus compositions or mixtures thereof is provided. The administration improves one or more of the performance characteristics described herein, such as: (a) increased survival, (b) increased body weight gain (either or both of average daily weight gain or total weight gain), (c) increased feed intake, (d) increased length, (e) feed conversion, which includes both feed:gain and gain:feed, (f) increased villi length and/or density, (g) increased resistance to low salinity, (h) increased resistance to high salinity, (i) increased resistance to high temperature, (j) increased resistance to low temperature, (k) increased resistance to formalin, (l) increased survival in response to a pathogenic agent, such as WSSv or Vibrio spp., or (m) mortality.

The following Examples are provided for illustrative purposes only. The Examples are included herein solely to aid in a more complete understanding of the presently described invention. The Examples do not limit the scope of the invention described or claimed herein in any fashion.

### EXAMPLES

### Example 1

Effect of Bacillus and Heat-Inactivated Lactobacillus on Growth Performance in White Shrimp (Penaeus vannamei) Post Larvae

In this study, initial weight, final weight, weekly weight gain, feed intake, feed conversion ratio (FCR), length, survival, muscle gut ratio, color of hepatopancreas, deformities, and degree of fouling were measured. White shrimp Litopenaeus vannamei in their post larval stage were used. Five replicates were conducted for each of the treatments shown in Table 1 below (Bac=Bacillus; HIL=Heat Inactivated Lactobacillus.) Approximately 300 shrimp per square meter were maintained in a 200 liter glass aquarium. The water was maintained at 29 degrees Celsius with salinity of 25 ppt. Approximately 20 percent of the water was exchanged per day.

TABLE 1

| No | Treatment | Dose tested (g/ton) |
|---|---|---|
| 1 | Control group | — |
| 2 | Bac 500 | 500 (10⁸ CFU/g of feed) |
| 3 | HIL 500 | 500 |
| 4 | HIL 1000 | 1000 |
| 5 | HIL 500 + Bac 500 | 500 g/ton HIL + 500 g/ton Bac |

Bacillus feed compositions comprised dried Bacillus fermentation product, calcium carbonate, rice hulls, and mineral oil. The minimal count was 2×10⁸-2×10⁹ CFU/g. The composition comprised Bacillus pumilis 3064 (50%), Bacillus subtilis BS 2084 (25%), and Bacillus subtilis BS15 Ap4 (25%). As discussed supra, Bacillus pumilus 3064 and Bacillus pumilis 119 were determined to be genetically equivalent based on RAPD banding patterns and cpn60 and 16S rDNA sequence analysis of five replicate samples. Accordingly Bacillus pumilus 3064 and Bacillus pumilis 119 are used interchangeably herein.

Lactobacillus feed compositions were heat inactivated. The initial concentration of Lactobacillus before heat inactivation was 8.10×10⁹ CFU/g. The theoretical concentration of Lactobacillus in the final product was 8.10×10⁸ CFU/g. Lactobacillus compositions comprised heat inactivated Lactobacillus, seaweed meal, crushed expanded corn, crushed soy (obtained by extraction), micronized bran, silicon dioxide, and calcium propionate. The compositions comprised Lactobacillus rhamnosus MA27/6B, Lactobacillus farciminis MA27/6R.

Commercial shrimp feed appropriated to shrimp size was employed. Bacillus and/or Lactobacillus cells were weighted according to dosage requirement, then mixed with 1.5% sterile normal saline (feed 1 g/125 microliter normal saline). This solution was mixed homogeneously with shrimp feed. The shrimp feeds were coated with fish oil by the ratio of fish oil 40 microliter/g feed. Samples were kept at −4 degrees Celsius until used. Bacillus and/or Lactobacillus cells were included in feed by top-dressing after post-extrusion.

Post larvae were fed every 4 h as routinely performed in the hatchery. The shrimp were fed with live artemia and change to trial feed (meal by meal/alternative feed sequence) until the end of the experimental period for 20 days. The amount of trial feed in each meal were recorded and carefully adjusted.

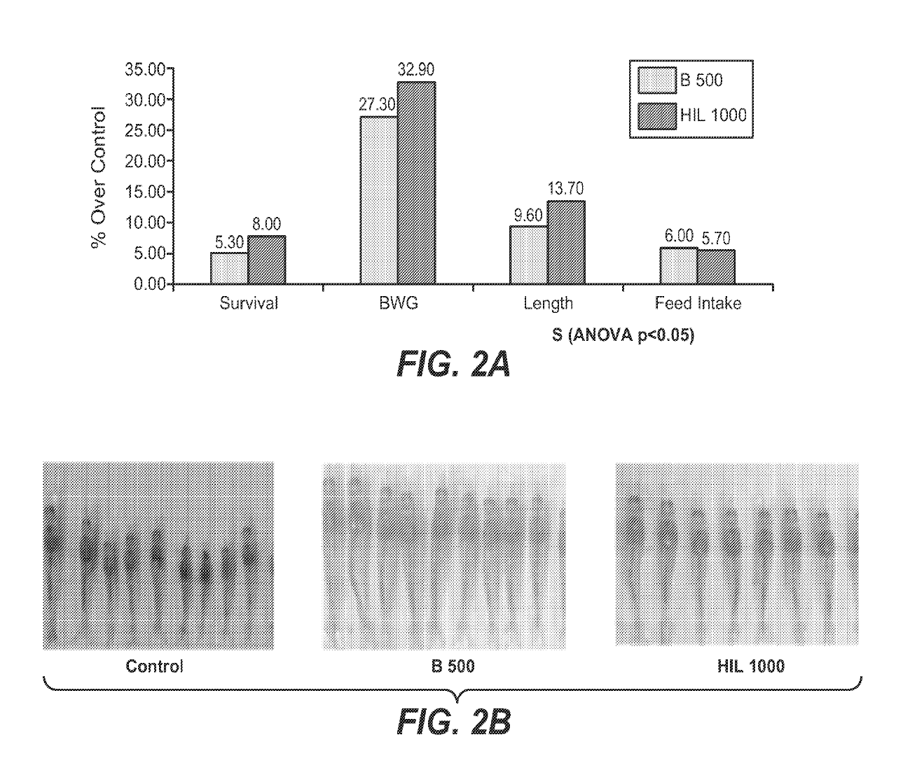

As seen in FIG. 2, administration of either Bacillus or Lactobacillus compositions resulted in increases in survival, body weight gain (BWG), length, and feed intake in post-larvae shrimp as compared to a control group of untreated shrimp. Administration of Bacillus compositions at 500 grams per ton of feed resulted in a 5.3 percent increase in survival, a 27.3 percent increase in body weight gain, a 9.6 percent increase in length, and a 6.0 percent increase in feed intake. Administration of Lactobacillus compositions at 1000 grams per ton of feed resulted in a 8.0 percent increase in survival, a 32.9 percent increase in body weight gain, a 13.7 percent increase in length, and a 5.7 percent increase in feed intake. All values are relative to a control group that did not receive the Bacillus or Lactobacillus composition, and are a summary of four trials (S (ANOVA p<0.05)).

### Example 2

Effect of Bacillus and Heat-Inactivated Lactobacillus on Stress Resistance in White Shrimp (Penaeus vannamei) Post Larvae

In this study, the stress response of post larvae shrimp to high salinity, low salinity, high temperature, and low temperature were evaluated. In addition, the stress response of the shrimp to formalin was evaluated. Survival, HSP70 heat shock protein, glutathione peroxidase (GPx), and N/K ATPase were measured. HSP70 is a 70 kDa heat shock protein that is a conserved molecular chaperone, found in the cytosol and in other compartments of the cell, that promotes the survival of stressed cells. They play an essential role in the life cycle of many proteins under both normal and stressful conditions. Glutathione peroxidase (GPx) is the general name of an enzyme family with peroxidase activity whose main biological role is to protect the organism from oxidative damage. The biochemical function of glutathione peroxidase is to reduce lipid hydroperoxides to their corresponding alcohols and to reduce free hydrogen peroxide to water. Formaldehyde solution (or formalin) is a general disinfectant used as a germicide, fungicide or preservative in various industries. Its main mode of action is to form covalent cross links with functional groups on proteins. In the context of aquaculture, it is used as a disinfectant in hatcheries.

White shrimp Litopenaeus vannamei in their post larvae stage were used. Five replicates were conducted for each of the treatments shown in Table 2 below (Bac=Bacillus; HIL=Heat Inactivated Lactobacillus.) Approximately 40 shrimp were maintained per 10 liter glass aquarium. Post larvae were stocked in brackish water of 20 ppt for the formalin stress test of 0 or 800 ppm. Post larvae were stocked in brackish water of 25 ppt of 15 degrees Celsius and 35 degrees Celsius for the temperature stress test. Salinity stress tests were conducted in 0-5 ppt and 40 ppt at room temperature of 27-28 degrees Celsius. The shrimp were exposed to stress for 24 hours.

TABLE 2

| No | Treatment | Dose tested (g/ton) | Stress Test |
|---|---|---|---|
| 1 | Control group | — | 0-5 and 40 ppt, |
| 2 | Bac 500 | 500 | 15° C. and 35° C. |
| 3 | HIL 500 | 500 | Formalin 800 ppm |
| 4 | HIL 1000 | 1000 | |
| 5 | HIL 500 + | 500 g/ton HIL + | |
| | Bac 500 | 500 g/ton Bac | |

Bacillus and Lactobacillus compositions, feed compositions, and diet preparation were prepared as described in Example 1. As seen in Table 3, administration of either Bacillus (500 grams per ton of feed) or Lactobacillus (1000 grams per ton of feed) resulted in increased resistance to stress in shrimp. In particular, administration of Bacillus (500 grams per ton of feed) or Lactobacillus (1000 grams per ton of feed) compositions resulted in dramatically increased resistance to low salinity (17.7 percent and 8.2 percent, respectively) and high salinity (1.1 percent and 6.5 percent, respectively). In addition, administration of Bacillus or Lactobacillus resulted in dramatically increased resistance to low temperature (12.5 percent and 12.7 percent, respectively). Administration of Lactobacillus also resulted in increased resistance to high temperature (3.2 percent). All values are relative to an untreated control population that did not receive the Bacillus or Lactobacillus composition.

These data are particularly relevant for aquaculture facilities that are located where there is a strong rainy season, such as Asia. Low salinity and low temperatures often occur during such rainy seasons.

TABLE 3

| Treatment | Bacillus (500 g/ton) | HIL (1000 g/ton) |
|---|---|---|
| Low salinity (0-5 ppt) | +17.7 | +8.2 |
| High salinity (40 ppt) | +1.1 | +6.5 |
| Low temperature (15 C.) | +12.5 | +12.7 |
| High temperature (35 C.) | −10.1 | +3.2 |

*Values shown are percent increase over an untreated control population S (ANOVA p < 0.05)

As seen in Table 4, administration of either Bacillus (500 grams per ton of feed) or Lactobacillus (1000 grams per ton of feed) resulted in increased activity of both pectinase and amylase in the hepatopancreas and intestine of juvenile shrimp. In particular, administration of Bacillus or Lactobacillus compositions resulted in dramatically increased activity of pectinase in hepatopancreas (30.2 percent and 19.6 percent, respectively) and intestines (0.2 percent and 6.11 percent, respectively). Administration of Bacillus or Lactobacillus compositions resulted in dramatically increased activity of amylase in hepatopancreas (4.8 percent and 13.1 percent, respectively) and intestines (6.8 percent and 10.2 percent, respectively). All values are relative to an untreated control population that did not receive the Bacillus or Lactobacillus composition.

TABLE 4

| | Bacillus (500 g/ton) | HIL (1000 g/ton) |
|---|---|---|
| Relative pectinase activity | +30.2 | +19.6 |
| in hepatopancreas | | |
| Relative pectinase activity | +0.2 | +6.11 |
| in intestine | | |
| Relative amylase activity | +4.8 | +13.1 |
| in hepatopancreas | | |
| Relative amylase activity | +6.8 | +10.2 |
| in intestine | | |

*Values shown are percent increase over an untreated control population S (ANOVA p < 0.05)

### Example 3

Effect of Bacillus and Heat-Inactivated Lactobacillus on Growth Performance in Juvenile White Shrimp (Penaeus vannamei)

In this study, weight gain, length, feed conversion ratio (FCR), digestive enzymes (pectinase, amylase) in hepatopancreas and intestine, and gut histology were measured. Juvenile white shrimp Litopenaeus vannamei were tested. Five replicates were conducted for each of the treatments shown in Table 5 below (Bac=Bacillus; HIL=Heat Inactivated Lactobacillus.) In one study, approximately 25 shrimp per aquarium were tested per replicate. The water was maintained at 29 degrees Celsius with salinity of 25 ppt. In another study, approximately 300 shrimp were kept per net per replicate test. Shrimp were acclimatized in the net cages for seven days prior to the start of the experiment. The net cages were installed in a shrimp pond and covered with a net sheet to prevent the shrimp from escaping. Two sets of paddle wheels were equipped in the shrimp pond to increase the dissolved oxygen and to circulate the water.

TABLE 5

| No | Treatment | Dose tested (g/ton) |
|---|---|---|
| 1 | Control group | — |
| 2 | Bac 500 | 500 |
| 3 | HIL 500 | 500 |
| 4 | HIL 1000 | 1000 |
| 5 | HIL 500 + Bac 500 | 500 g/ton HIL + 500 g/ton Bac |

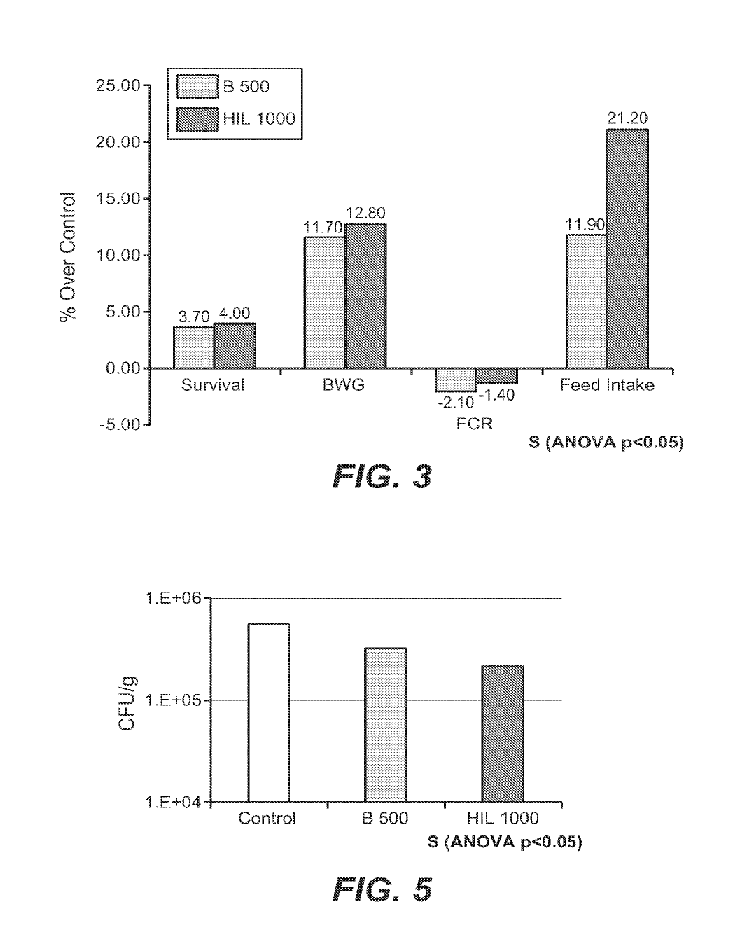

Bacillus and Lactobacillus compositions, feed compositions, and diet preparation were prepared as described in Example 1. As seen in FIG. 3, administration of either Bacillus (500 grams per ton of feed) or Lactobacillus (1000 grams per ton of feed) compositions resulted in increases in survival, body weight gain (BWG), and feed intake in juvenile shrimp. Administration of Bacillus compositions at 500 grams per ton of feed resulted in a 3.7 percent increase in survival, a 11.7 percent increase in body weight gain, and a 11.9 percent increase in feed intake. Administration of Lactobacillus compositions at 1000 grams per ton of feed resulted in a 4.0 percent increase in survival, a 12.8 percent increase in body weight gain, and a 21.2 percent increase in feed intake. All values are relative to a control group that did not receive the Bacillus or Lactobacillus composition, and are a summary of three trials (S (ANOVA p<0.05)).

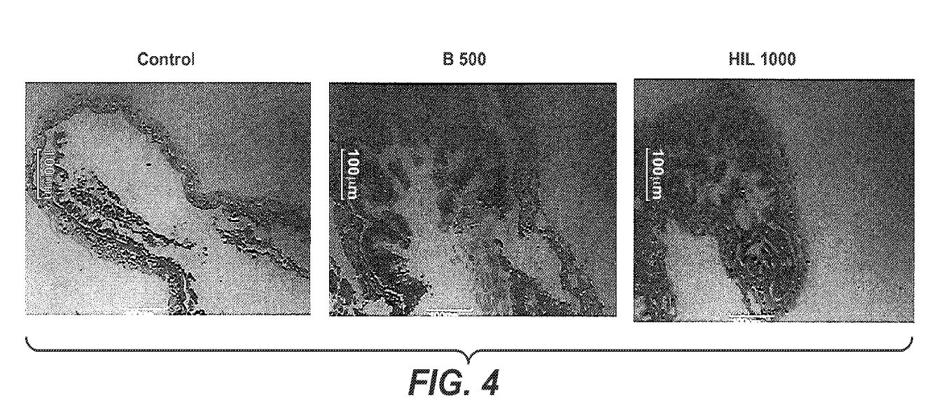

At the end of the study, the histology of the villi in the shrimp was evaluated. The increased performance of shrimp administered with Bacillus or Lactobacillus compositions appears, at least in part, to be due to improved gut physiology. As seen in FIG. 4, shrimp fed a diet supplemented with Bacillus or Lactobacillus exhibited longer and higher density villi than a control group that was not feed the supplement. This improved gut physiology suggests a higher potential for digestion in the shrimp that are administered Bacillus or Lactobacillus compositions.

### Example 4

Effect of Bacillus and Heat-Inactivated Lactobacillus on Stress Resistance in Juvenile White Shrimp (Penaeus vannamei)

In this study, the stress response to high salinity, low salinity, high temperature, and low temperature were evaluated. In addition, the stress response to formalin was evaluated. Survival, HSP70 heat shock protein, glutathione peroxidase (GPx), lipid peroxidase, TBAR, and catalase were measured.

Salinity: Twenty shrimp from three replicates of treatments in Table 6 were sampled for study on salinity stress test by division into two groups. The first group was stocked in a glass aquarium with 40 ppt water. The second group was stocked in a glass aquarium with 0-5 ppt water. The survival rate was evaluated.

Formalin: Twenty shrimp from three replicates of treatments in Table 6 were sampled for study on formalin stress test by division into two groups. The first group was stocked in 10 L glass aquarium with 0 ppm formalin in water. The second group was stocked in 10 L glass aquarium with 600 ppm formalin in water. The survival rate was evaluated everyday for one week.

Temperature: 20 shrimp from each replicate was sampled every week during 30 days for study on temperature stress tolerance. The first group was stocked in small basket hang in 1,000 L tank with 35° C. water. Shrimp from each treatment was stocked 24 hr. The second group was stocked in 10 L glass aquarium with 15° C. water for study on the temperature stress tolerance. Shrimp from each treatment was stocked for one hour.

TABLE 6

| No | Treatment | Dose tested (g/ton) | Stress Test |
|---|---|---|---|
| 1 | Control group | — | 0-5 and 40 ppt, |
| 2 | Bac 500 | 500 | 15° C. and 35° C. |
| 3 | HIL 500 | 500 | Formalin 800 ppm |
| 4 | HIL 1000 | 1000 | |
| 5 | HIL 500 + | 500 g/ton HIL + | |
| | Bac 500 | 500 g/ton Bac | |

Bacillus and Lactobacillus compositions, feed compositions, and diet preparation were prepared as described in Example 1. As seen in Table 7, administration of either Bacillus (500 grams per ton of feed) or Lactobacillus (1000 grams per ton of feed) resulted in increased resistance to stress in juvenile shrimp. In particular, administration of Bacillus or Lactobacillus compositions resulted in dramatically increased resistance to low salinity (13.3 percent and 10.0 percent, respectively). Administration of Bacillus compositions also resulted in increased resistance to high salinity (1.8 percent). In addition, administration of Bacillus or Lactobacillus compositions resulted in dramatically increased resistance to low temperature (16.4 percent and 16.4 percent, respectively). Administration of Bacillus compositions also resulted in increased resistance to high temperature (3.3 percent). All values are relative to an untreated control population that did not receive the Bacillus or Lactobacillus composition.

As discussed in previous examples, these data are particularly relevant for aquaculture facilities that are located where there is a strong rainy season, such as Asia. Low salinity and low temperatures often occur during such rainy seasons.

TABLE 7

| Treatment | Bacillus (500 g/ton) | HIL (1000 g/ton) |
|---|---|---|
| Low salinity (0-5 ppt) | +13.3 | +10.0 |
| High salinity (40 ppt) | +1.8 | −0.7 |
| Low temperature (15 C.) | +16.4 | +16.4 |
| High temperature (35 C.) | +3.3 | −5.0 |

*Values shown are percent increase over an untreated control population S (ANOVA p < 0.05)

### Example 5

Effect of Bacillus and Heat-Inactivated Lactobacillus on Disease Response in White Shrimp (Penaeus vannamei)

In this study, survival, immune parameters (e.g., total hemocyte count (THC), phagocytic activity, glucose level, oxyhemocyanin (Oxy), ratio of oxyhemocyanin:protein (Oxy:prot), phenoloxidase activity (PO), and hemolymph protein), and gene expression of proPO, HSP70, SP, PE, and LGBPP were measured. White shrimp Litopenaeus vannamei were measured for their disease response. Five replicates were conducted for each of the treatments shown in Table 8 below (Bac=Bacillus; HIL=Heat Inactivated Lactobacillus.) Approximately 20-25 shrimp per aquarium were tested per replicate.

WSSv disease challenge: At the end of the experiment, shrimp from each treatment were tested for disease resistance against White Spotted Syndrome virus (WSSv) infection. Shrimp were injected with WSSv suspension at the concentration of LD₅₀ which was previously determined. Mortality was recorded for 10 days post challenge. The causative mortality was confirmed by PCR analysis.

Vibrio challenge: At the end of the experiment, shrimp from each treatment were tested for disease resistance against V. harveyi infection. A bacterial suspension of V. harveyi was prepared from a 18-24 hour culture and was adjusted to reach a final concentration of approximately 10⁶ CFU/ml of culture water. After exposure, shrimp were moved back to culture tanks and the mortality was recorded for 14 days. Total Vibrio spp. in cultured water and shrimp intestine counts were performed using Thiosulphate Citrate Bilesalt Sucrose as a specific culture media for Vibrionaceae. Total Vibrio spp. were calculated after incubation at 35 degrees Celsius for 18-24 hours.

TABLE 8

| No | Treatment | Dose tested (g/ton) |
|---|---|---|
| 1 | Control group | — |
| 2 | Bac 500 | 500 |
| 3 | HIL 1000 | 1000 |
| 4 | HIL 500 + Bac 500 | 500 g/ton HIL + 500 g/ton Bac |

Bacillus and Lactobacillus compositions, feed compositions, and diet preparation were prepared as described in Example 1. As seen in Table 9, administration of either Bacillus (500 grams per ton of feed) or Lactobacillus (1000 grams per ton of feed) resulted in increased survival in shrimp 10-14 days after exposure to either WSSv or Vibrio. In particular, administration of Bacillus (500 grams per ton of feed) or Lactobacillus (1000 grams per ton of feed) compositions resulted in dramatically increased survival in response exposure to WSSv (6.1 percent and 12.2 percent, respectively) and Vibrio (8.8 percent and 2.3 percent, respectively). Furthermore, as seen in FIG. 5, administration of Bacillus (500 grams per ton of feed) or Lactobacillus (1000 grams per ton of feed) compositions resulted in a decrease in the amount of pathogens in the shrimp gut (S (ANOVA p<0.05)).

All values are relative to an untreated control population that did not receive the Bacillus or Lactobacillus composition (S (ANOVA p<0.05)).

TABLE 9

| | Bacillus (500 g/ton) | HIL (1000 g/ton) |
|---|---|---|
| WSSv exposure | +6.1 | +12.2 |
| Vibrio exposure | +8.8 | +2.3 |

*Values shown are percent increase over an untreated control population S (ANOVA p < 0.05)

Administration of either Bacillus (500 grams per ton of feed) or Lactobacillus (1000 grams per ton of feed) compositions appears to protect shrimp against WSSv and Vibrio pathogens at least partially due to the stimulation of expression of genes and cellular activities involved in immunity process. For example, phagocytic activity was increased by 21.0 percent when shrimp were treated with Bacillus compositions, relative to an untreated control. Furthermore, phenoloxidase activity increased by 24.8 percent when shrimp were treated with Lactobacillus compositions, relative to an untreated control. In addition, proPO and HSP70 gene expression were decreased in WSSv exposed shrimp that were treated with Bacillus or Lactobacillus compositions.

### Example 6

Improved Performance in White Shrimp (Penaeus vannamei) Compared to Alternative Commercial Bacillus-Based Solutions

The present Bacillus compositions were compared to alternative commercial Bacillus-based solutions (Novozymes PondPlus® and INVE Sanolife) for their effect on growth performance and mortality in white shrimp (Penaeus vannamei) grown in outdoor ponds. Three replicates were conducted for each of the treatments shown in Tables 10 and 13 below. Outdoor ponds were approximately 3330 square meters in size and were stocked with approximately 187,000 shrimp/pond (562,500 shrimp/hectare). Prior to initiation of the trials, shrimp underwent a two week conditioning period during which they readily adjusted to the basal diet and experimental conditions. After the conditioning period, shrimp were fed twice per day to apparent satiation during the five month trial. The water temperature varied from 20-31 degrees Celsius during the trial period. All diets were isonitrogenous and isoenergetic (See Table 11). Feed was provided by the Zhejiang Xinxin Feed Co., Ltd (Jiaxin, Zhejiang, China). Each pond was provided with two aerators, with no water discharge. Individual body weight was measured in the middle and end of the trial. Feed intake was recorded daily. Stocking density (shrimp/hectare), harvest weight (kg/hectare), feed intake (kg/hectare), feed conversion ratio (FCR) and survival rate (%) were also measured.

All data were expressed as means±SD. The data were analyzed by one-way ANOVA. Differences among groups were analyzed using Duncan's procedure. Differences with a P<0.05 were considered statistically significant. All tests were performed using SPSS 11.5.

TABLE 10

| No | Treatment | Dose tested (g/ton) |
|---|---|---|
| 1 | Control | — |
| 2 | Bacillus subtilis BS2084 | In feed application |
| | Bacillus subtilis BS 15Ap4 | 50 g/ton of feed |
| | Bacillus pumilis BP119 | (2 × 10⁹ CFU/g of feed) |
| 3 | Microsource | In feed application |
| | Bacillus subtilis BS27 | 50 g/ton of feed |
| | Bacillus licheniformis BA842 | (2 × 10⁹ CFU/g of feed) |
| | Bacillus licheniformis BL21 | |
| 4 | Novozymes PondPlus ® | In water application |
| | | 1^{st} 60 days: 20-25 g |
| | | (>10 × 10⁸ CFU/g) |
| | | per 333 m² every 7 days |
| | | >60 days: 25-30 g |
| | | (>10 × 10⁸ CFU/g) |
| | | per 333 m² every 7 days |

Bacillus feed compositions in Treatments 2 and 3 comprised dried Bacillus fermentation product, calcium carbonate, rice hulls, and mineral oil. The composition was incorporated into shrimp feed by spraying a liquid Bacillus solution onto the surface of the shrimp feed. Novozymes PondPlus® was sprayed directly into the pond.

As discussed supra, Bacillus pumilus 3064 and Bacillus pumilis 119 were determined to be genetically equivalent based on RAPD banding patterns and cpn60 and 16S rDNA sequence analysis of five replicate samples. Accordingly Bacillus pumilus 3064 and Bacillus pumilis 119 are used interchangeably herein.

TABLE 11

| | Nutrient composition (%) |
|---|---|
| Crude protein | 43.19 ± 0.06 |
| Crude lipid | 6.22 ± 0.35 |
| Ash | 10.91 ± 0.05 |

TABLE 12
Growth Performance

| | | Stocking density | Harvest weight | Feed intake | | Survival rate |
|---|---|---|---|---|---|---|
| No | | (shrimp/hectare) | (kg/hectare) | (kg/hectare) | FCR | (%) |
| 1 | Control | 56.25 × 10⁴ | 2892.50 ± 38.26^{c} | 4907.38 ± 139.75^{a} | 1.69 ± 0.05^{a} | 42.33 ± 0.6^{b} |
| 2 | Novozymes PondPlus ® | 56.25 × 10⁴ | 3015.50 ± 79.15^{bc} | 4383.09 ± 179.99^{b} | 1.45 ± 0.04^{b} | 44.00 ± 1.7^{ab} |
| 3 | Microsource | 56.25 × 10⁴ | 3104.25 ± 93.16^{b} | 4334.87 ± 177.44^{b} | 1.40 ± 0.05^{b} | 44.00 ± 1.0^{ab} |
| | Bacillus subtilis BS27 | | | | | |
| | Bacillus licheniformis BA842 | | | | | |
| | Bacillus licheniformis BL21 | | | | | |
| 4 | Bacillus subtilis BS2084 | 56.25 × 10⁴ | 3446.00 ± 60.25^{a} | 4409.73 ± 38.47^{b} | 1.28 ± 0.03^{c} | 45.33 ± 1.2^{a} |
| | Bacillus subtilis 15Ap4 | | | | | |
| | Bacillus pumilis BP119 | | | | | |

As seen in Table 12, all treatments increased the final production yield of white shrimp in pond in terms of final weight and FCR (p<0.05) when compared with untreated control treatment. Survival rate was also increased by all treatments. Notably, the Bacillus subtilis BS2084, Bacillus subtilis BS 15Ap4, and Bacillus pumilis BP119 composition (Treatment 4) and the Microsource® Bacillus subtilis BS27, Bacillus licheniformis BA842 and Bacillus licheniformis BL21 compositions (Treatment 3) showed significant improvements in growth performance compared to Novozymes Pondplus® Bacillus product. The Bacillus subtilis BS2084, Bacillus subtilis BS 15Ap4, and Bacillus pumilis BP119 composition showed particularly significant improvements in growth performance, demonstrating a 19.2% increase in harvest weight when compared to the untreated control sample, and a 14.3% increase in harvest weight when compared to Novozymes Pondplus®. The Bacillus subtilis BS2084, Bacillus subtilis BS 15Ap4, and Bacillus pumilis BP119 composition showed an improvement of 24.3% in FCR when compared to the untreated control treatment, and an improvement of 11.7% when compared to Novozymes Pondplus®. The Microsource® Bacillus subtilis BS27, Bacillus licheniformis BA842 and Bacillus licheniformis BL21 composition also demonstrated significant improvements over the untreated control and Novozymes Pondplus® (See Table 12).

TABLE 13

| No | Treatment | Dose tested (g/ton) |
|---|---|---|
| 1 | Control Group | — |
| 2 | Microsource | In feed application |
| | Bacillus subtilis BS27 | 50 g/ton of feed |
| | Bacillus licheniformis BA842 | (2 × 10⁹ CFU/g of feed) |
| | Bacillus licheniformis BL21 | |
| 3 | INVE Sanolife | |

Bacillus feed compositions in Treatment 2 of Table 13 comprised dried Bacillus fermentation product, calcium carbonate, rice hulls, and mineral oil. The composition was incorporated into shrimp feed by spraying a liquid Bacillus solution onto the surface of the shrimp feed.

TABLE 14
Growth Performance

| | | Stocking density | Harvest weight | Feed intake | | Survival rate |
|---|---|---|---|---|---|---|
| No | | (shrimp/hectare) | (kg/hectare) | (kg/hectare) | FCR | (%) |
| 1 | Control | 56.25 × 10⁴ | 2672.75 ± 79.14 | 4805.44 ± 328.51 | 1.79 ± 0.07 | 40.86 ± 2.01 |
| 2 | INVE Sanolife | 56.25 × 10⁴ | 2833.50 ± 57.07 | 4400.75 ± 97.57 | 1.55 ± 0.04 | 40.26 ± 1.02 |
| 3 | Microsource | 56.25 × 10⁴ | 3145.25 ± 15.30 | 4539.90 ± 127.59 | 1.44 ± 0.04 | 43.61 ± 0.10 |
| | Bacillus subtilis BS27 | | | | | |
| | Bacillus licheniformis BA842 | | | | | |
| | Bacillus licheniformis BL21 | | | | | |

As seen in Table 14, compared with untreated control treatment, Treatments 2 and 3 increased the final production yield of white shrimp in pond in terms of final weight and FCR (p<0.05). Notably, Microsource® Bacillus subtilis BS27, Bacillus licheniformis BA842 and Bacillus licheniformis BL21 composition (Treatment 3) showed a significant improvement in growth performance compared to INVE Sanolife. Microsource® Bacillus subtilis BS27, Bacillus licheniformis BA842 and Bacillus licheniformis BL21 composition increased harvest weight by 17.7% over the untreated control and by 11.0% over the INVE Sanolife sample. Microsource® Bacillus subtilis BS27, Bacillus licheniformis BA842 and Bacillus licheniformis BL21 composition improved FCR by 19.6% over the untreated control and by 7.1% over the INVE Sanolife sample. Survival rate was also increased with Microsource® Bacillus subtilis BS27, Bacillus licheniformis BA842 and Bacillus licheniformis BL21 composition when compared to both the untreated control and the INVE Sanolife sample.

These results demonstrate that the present Bacillus compositions are significantly superior to alternative microbiological solutions for increasing the growth performance and production yield of white shrimp farmed in pond when used as a feed additive. Final weight gain and FCR were clearly improved when the present compositions were compared to both Novozymes Pondplus® and INVE Sanolife, as well us untreated control treatments.

What is claimed is:

1. A composition comprising one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

2. The composition of claim 1, wherein the composition comprises a Bacillus pumilis 3064 strain, a Bacillus subtilis BS 2084 (NRRL B-50013) strain, and a Bacillus subtilis BS15 Ap4 (ATCC PTA-6507) strain.

3. The composition of claim 1, wherein the composition comprises a Bacillus pumilis 119 (NRRL B-50796) strain, a Bacillus subtilis BS 2084 (NRRL B-50013) strain, and a Bacillus subtilis BS15 Ap4 (ATCC PTA-6507) strain.

4. The composition of claim 1, wherein the composition comprises a Bacillus subtilis 1013 (NRRL B-50509) strain, a Bacillus subtilis BS918 (NRRL B-50508) strain, and a Bacillus subtilis BS3BP5 (ATCC PTA-50510) strain.

5. The composition of claim 1, wherein the composition comprises a Bacillus licheniformis 842 (NRRL B-50516) strain, a Bacillus subtilis BS27 (NRRL B-50105) strain, and a Bacillus licheniformis BL21 (ATCC PTA-50134) strain.

6. The composition of claim 1, wherein the composition comprises a Bacillus subtilis 3A-P4 (ATCC PTA-6506), strain, a Bacillus subtilis BS15 Ap4 (ATCC PTA-6507) strain, and a Bacillus subtilis 22C-P1 (ATCC PTA-6508) strain.

7. A method comprising administering to an animal an effective amount of a composition comprising one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

8. The method of claim 7, wherein upon administration to the animal, the strain provides at least one of the following benefits in or to the animal when compared to an animal not administered the strain: (a) increased survival, (b) increased body weight gain (either or both of average daily weight gain or total weight gain), (c) increased feed intake, (d) increased length, (e) increased feed conversion, (f) increased villi length and/or density, (g) increased resistance to low salinity, (h) increased resistance to high salinity, (i) increased resistance to high temperature, (j) increased resistance to low temperature, (k) increased resistance to formalin, (l) increased survival in response a pathogenic agent, or (m) mortality.

9. The method of any one of claims 7 or 8, wherein the animal is a shrimp.

10. The method of any one of claims 7-9, wherein the animal is a larvae, post-larvae, or juvenile shrimp.

11. The method of any one of claims 7-10, wherein the animal is Penaeus vannamei.

12. The method of any one of claims 7-11, wherein, when the strain is administered to the animal, the strain provides an improvement in at least one of the benefits by at least 2% compared to a control.

13. The method of any one of claims 7-12, wherein the strain(s) is/are administered at about 1×10⁵ to about 1×10¹¹ CFU/animal/day.

14. The method of any one of claims 7-13, wherein the pathogenic agent is White Spot Syndrome virus or Vibrio spp.

15. The method of any one of claims 7-14, wherein the pathogenic agent is White Spot Syndrome virus or Vibrio harveyi.

16. The method of any one of claims 7-15, wherein the animal is exposed to high or low temperatures, high or low salinity, white spotted syndrome virus, or Vibrio spp.

17. The method of any one of claims 7-16, wherein the composition comprises a Bacillus pumilis 3064 strain. a Bacillus subtilis BS 2084 (NRRL B-50013) strain, and a Bacillus subtilis BS15 Ap4 (ATCC PTA-6507) strain.

18. The method of any one of claims 7-16, wherein the composition comprises a Bacillus pumilis 119 (NRRL B-50796) strain, a Bacillus subtilis BS 2084 (NRRL B-50013) strain, and a Bacillus subtilis BS15 Ap4 (ATCC PTA-6507) strain.

19. The method of any one of claims 7-16, wherein the composition comprises a Bacillus subtilis 1013 (NRRL B-50509) strain, a Bacillus subtilis BS918 (NRRL B-50508) strain, and a Bacillus subtilis BS3BP5 (ATCC PTA-50510) strain.

20. The method of any one of claims 7-16, wherein the composition comprises a Bacillus licheniformis 842 (NRRL B-50516) strain, a Bacillus subtilis BS27 (NRRL B-50105) strain, and a Bacillus licheniformis BL21 (ATCC PTA-50134) strain.

21. The method of any one of claims 7-16, wherein the composition comprises a Bacillus subtilis 3A-P4 (ATCC PTA-6506), strain, a Bacillus subtilis BS15 Ap4 (ATCC PTA-6507) strain, and a Bacillus subtilis 22C-P1 (ATCC PTA-6508) strain.

22. An animal feed or feed additive composition, comprising one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof.

23. The composition of claim 22, wherein the one or more strains is supplemented in the composition in an amount of 10 to 2000 grams per ton of feed.

24. The composition of claim 22 or 23, wherein the composition comprises a Bacillus pumilis 3064 strain, a Bacillus subtilis BS 2084 (NRRL B-50013) strain, and a Bacillus subtilis BS15 Ap4 (ATCC PTA-6507) strain.

25. The composition of claim 22 or 23, wherein the composition comprises a Bacillus pumilis 119 (NRRL B-50796) strain, a Bacillus subtilis BS 2084 (NRRL B-50013) strain, and a Bacillus subtilis BS15 Ap4 (ATCC PTA-6507) strain.

26. The composition of claim 22 or 23, wherein the composition comprises a Bacillus subtilis 1013 (NRRL B-50509) strain, a Bacillus subtilis BS918 (NRRL B-50508) strain, and a Bacillus subtilis BS3BP5 (ATCC PTA-50510) strain.

27. The composition of claim 22 or 23, wherein the composition comprises a Bacillus licheniformis 842 (NRRL B-50516) strain, a Bacillus subtilis BS27 (NRRL B-50105) strain, and a Bacillus licheniformis BL21 (ATCC PTA-50134) strain.

28. The composition of claim 22 or 23, wherein the composition comprises a Bacillus subtilis 3A-P4 (ATCC PTA-6506) strain, a Bacillus subtilis BS15 Ap4 (ATCC PTA-6507) strain, and a Bacillus subtilis 22C-P1 (ATCC PTA-6508) strain.

29. A method of producing one or more isolated strains selected from the group consisting of Bacillus pumilis 3064, Bacillus subtilis BS 2084 (NRRL B-50013), Bacillus subtilis BS15 Ap4 (ATCC PTA-6507), Bacillus subtilis AGTP BS3BP5 (NRRL B-50510), Bacillus subtilis AGTP BS442 (NRRL B-50542), Bacillus subtilis AGTP BS521 (NRRL B-50545), Bacillus subtilis AGTP BS918 (NRRL B-50508), Bacillus subtilis AGTP BS1013 (NRRL B-50509), Bacillus pumilis 119 (NRRL B-50796), Bacillus subtilis 3A-P4 (ATCC PTA-6506), Bacillus subtilis 22C-P1 (ATCC PTA-6508), Bacillus licheniformis 842 (NRRL B-50516), Bacillus subtilis BS27 (NRRL B-50105), Bacillus licheniformis BL21 (NRRL B-50134), Bacillus pumilus AGTP BS 1068 (NRRL B-50543), and Bacillus subtilis AGTP BS1069 (NRRL B-50544), Lactobacillus farcimins CNCM-I-3699, and Lactobacillus rhamnosus CNCM-I-3698, and strains having all the characteristics thereof, any derivative or variant thereof, and mixtures thereof, comprising: (a) growing, in a liquid broth, a culture including the one or more strain(s); and (b) separating the one or more strains from the liquid broth.

30. The method of claim 29, further comprising freeze drying the isolated strain and adding the freeze-dried strain to a carrier.

31. The method of claim 29 or 30, further comprising retaining the liquid broth after the strain has been separated from it to generate a supernatant.


SEQUENCE LISTING (6 sequences)

SEQ ID NO: 1 - DNA, 10 bp, Artificial Sequence
  source: /note=“Description of Artificial Sequence       Synthetic primer”
ggtgcgggaa

SEQ ID NO: 2 - DNA, 10 bp, Artificial Sequence
  source: /note=“Description of Artificial Sequence       Synthetic primer”
gtttcgctcc

SEQ ID NO: 3 - DNA, 10 bp, Artificial Sequence
  source: /note=“Description of Artificial Sequence       Synthetic primer”
gtagacccgt

SEQ ID NO: 4 - DNA, 10 bp, Artificial Sequence
  source: /note=“Description of Artificial Sequence       Synthetic primer”
aagagcccgt

SEQ ID NO: 5 - DNA, 10 bp, Artificial Sequence
  source: /note=“Description of Artificial Sequence       Synthetic primer”
aacgcgcaac

SEQ ID NO: 6 - DNA, 10 bp, Artificial Sequence
  source: /note=“Description of Artificial Sequence       Synthetic primer”
cccgtcagca